(19) United States
(12) Patent Application Publication
(10) Pub. No.: US 2002/0122816 A1
(43) Pub. Date: Sep. 5, 2002

(54) Use of 3-hydroxypropinoaldehyde in crosslinking and sterilizing a biomolecule, and a biocompatible implant, substitute or wound dressing containing the crosslinked biomolecule

(76) Inventors: Hsing-Wen Sung, Hsinchu (TW); Ching-Kuan Lin, Taichung (TW)

Correspondence Address: BACON & THOMAS, 4th Floor, 625 Slaters Lane, Alexandria, VA, 22314, US

(21) Appl. No.: 09/737,482

(22) Filed: Dec. 18, 2000

(51) Int. Cl. A61K 9/70; A61K 38/39; A61K 31/722

(52) U.S. Cl. 424/445; 514/12; 514/55

(57) ABSTRACT

A use of 3-hydroxypropinoaldehyde in the manufacture of a biocompatible implant, substitute or wound dressing is disclosed, which involves crosslinking an amine-containing biomolecule including chitosan, hemoglobin and a connective-tissue protein such as collagen or gelatin derived from a collagenous source with 3-hydroxypropinoaldehyde.

### FIELD OF THE INVENTION

The present invention is related to a biocompatible implant, substitute or wound dressing, and in particular to a biocompatible implant, substitute or wound dressing comprising a crosslinked biomolecule formed by crosslinking an amine-containing biomolecule with 3-hydroxypropinoaldehyde.

### BACKGROUND OF THE INVENTION

Axelsson and co-workers reported the discovery of a broad-spectrum antimicrobial reagent termed reuterin (3-hydroxypropinoaldehyde) produced by Lactobacillus reuteri [Axelsson, L., Chung, T. C., Dobrogosz, W. J., Lindgren, L. E., “Discovery of a new antimicrobial substance produced by Lactobacillus reuteri,” FEMS microbiol. rev., 46, 65, 1987]. Lactobacillus reuteri resides in the gastrointestinal tract of humans and animals. Cultures of Lactobacillus reuteri have been shown to accumulate large quantities of reuterin during anaerobic growth in the presence of glycerol [Axelsson, L., Chung, T. C., Dobrogosz, W. J., Lindgren, L. E., “Production of a broad spectrum antimicrobial substance by Lactobacillus reuteri,” Microb. ecol., 2, 131-136, 1989; Talarico, T. L., Dobrogosz, W. J., “Production and isolation of reuterin, a growth inhibitior produced by Lactobacillus reuteri,” Antimicrob. agents chemother., 32, 1854-1858, 1988]. Preliminary investigations indicate that it is a low-molecular-weight, neutral, water-soluble substance which has antibacterial, antimycotic, and antiprotozoal activity [Axelsson, L., Chung, T. C., Dobrogosz, W. J., Lindgren, L. E., “Production of a broad spectrum antimicrobial substance by Lactobacillus reuteri,” Microb. ecol., 2, 131-136, 1989].

Various fixatives including formaldehyde, glutaraldehyde, dialdehyde starch, and epoxy compound have been used in fixing biological tissues. Clinically, the most commonly used fixative is glutaraldehyde [Nimni, M. E., Cheung, D., Strates, B., Kodama, M., Sheikh, K., “Bioprosthesis derived from cross-linked and chemically modified collagenous tissues,” in Collagen Vol. III, M. E. Nimni (ed.), CRC Press, Boca Raton, Fla., 1988, pp.1-38]. Glutaraldehyde-fixed biological tissues have been used extensively to fabricate prosthetic heart valve prostheses, pericardial patches, vascular grafts, and ligament substitutes. However, the tendency for glutaraldehyde to markedly alter tissue stiffness and promote tissue calcification are well recognized drawbacks of this fixative.

To overcome the aforementioned deficiencies with the glutaraldehyde-fixed bioprostheses, the inventors of the present application and a co-worker developed a new fixation technique using genipin to fix biological tissues in the PCT patent application publication number WO 98/19718, wherein a biocompatible cross-linked materials, suitable for use in implants, wound dressings, and blood substitutes was provided. The materials are prepared by crosslinking biological substances, such as collagen, chitosan, or hemoglobin, with genipin, a naturally occurring crosslinking agent. The crosslinking agent has much lower toxicity than conventionally used reagents, and the cross-linked products have good thermal and mechanical stability as well as biocompatibility. The disclosure of this PCT patent application is incorporated herein by reference.

It is clear that there is still a need in the bio-technology industries for searching a crosslinking agent (fixative) for biological tissues having an improved performance in biocompatibility, cytotoxicity, and mechanical stability, and preferably having an additional sterilization effect.

### SUMMARY OF THE INVENTION

The present invention provides a biocompatible implant, substitute or wound dressing comprising a crosslinked biomolecule formed by crosslinking an amine-containing biomolecule with 3-hydroxypropinoaldehyde.

In an aspect of the present invention, an improved method of manufacturing a biocompatible implant, substitute or wound dressing is provided, wherein the improvement comprises crosslinking an amine-containing biomolecule with 3-hydroxypropinoaldehyde.

In another aspect of the present invention, a method for treating a patient requiring tissue prosthesis is disclosed, which comprises crosslinking an amine-containing biomolecule with 3-hydroxypropinoaldehyde, and implanting a biocompatible implant or wound dressing comprising the resulting crosslinked biomolecule into said patient.

In a further aspect of the present invention, a method for conducting blood transfusion in a patient is disclosed, which comprises crosslinking hemoglobin with 3-hydroxypropinoaldehyde and transfusing a blood substitute comprising the resulting crosslinked hemoglobin to said patient.

Preferably, said biomolecule is a connective-tissue protein, and more preferably is collagen or gelatin derived from a collagenous source

Preferably, said biomolecule is chitosan.

Preferably, said biomolecule is hemoglobin.

Preferably, said crosslinking in the methods of the present invention comprises contacting said biomolecule and 3-hydroxypropinoaldehyde in an aqueous medium at a temperature ranging from 4° C. to 50° C., preferably from 25° C. to 45° C., for a period ranging from 5 hours to 60 hours, preferably about 48 hours.

Preferably, said aqueous medium has a concentration of 3-hydroxypropinoaldehyde ranging from 0.01 M to 1.0 M, and more preferably from 0.03 M to 0.2 M Preferably, said aqueous medium has a pH value ranging from 3 to 12, and more preferably from 4 to 10.5.

### BRIEF DESCRIPTION OF THE DRAWINGS

FIGS. 1a and 1b show optical density (O.D.) readings of the 3T3 fibroblasts cultured in the media drugged with varying concentrations of glutaraldehyde (FIG. 1a) and reuterin (FIG. 1b) obtained in the MTT assay. The MTT₅₀ concentration was determined as the concentration of the test reagent required to reduce the optical density reading to half that of the control.

FIGS. 2a and 2b show fixation indices (FIG. 2a) and denaturation temperatures (FIG. 2b) of the glutaraldehyde- or reuterin-fixed tissues obtained at distinct elapsed fixation duration periods, wherein the rectangular dots represent the glutaraldehyde-fixed tissues and the round dots represent the reuterin-fixed tissues.

FIGS. 3a and 3b show fixation indices (FIG. 3a) and denaturation temperatures (FIG. 3b) of the tissues fixed by reuterin at different pHs

FIGS. 4a and 4b show fixation indices (FIG. 4a) and denaturation temperatures (FIG. 4b) of the tissues fixed by reuterin at different temperatures.

FIGS. 5a and 5b show fixation indices (FIG. 5a) and denaturation temperatures (FIG. 5b) of the tissues fixed by reuterin at different initial fixative concentrations.

### DETAILED DESCRIPTION OF THE INVENTION

Reuterin has antibacterial, antimycotic, and antiprotozoal activities as described in the articles mentioned in the Background of the Invention. Additionally, it is found by us that reuterin, 3-hydroxypropinoaldehyde, can react with the free amino groups within biological tissues. Therefore, reuterin can be used as a crosslinker (fixative) and a sterilant for biological tissues, natural products, or synthetic polymers in clinical applications. Reuterin has the following chemical structure:

HO—CH₂—CH₂—CH═O

and can be produced by Lactobacillus reuteri under control conditions. Reuterin used in following examples was identified by high performance liquid chromatography (HPLC).

Antimicrobial activity of reuterin was studied in the present invention, wherein glutaraldehyde was used as a control. The microorganisms tested in the study were Escherichia coli (ATCC 25922), Pseudomonas aeruginosa (ATCC 27853), Staphylococcus aureus (ATCC 25923), and Bacillus subtillis (ATCC 6633). The results show that all tested microorganisms, including both the gram-positive bacteria (Staphylococcus aureus and Bacillus subtillis) and gram-negative bacteria (Escherichia coli and Pseudomonas aeruginosa), were sensitive to reuterin. Generally, 20 to 35 ppm of reuterin can prevent the growth of the tested microorganisms, while 40 to 50 ppm of reuterin resulted in the death of the tested microorganisms. However, the values for glutaraldehyde were significantly greater than those for reuterin (approximately 2-3 times higher). This indicated that the antimicrobial activity of reuterin is significantly superior to glutaraldehyde.

The cytotoxicity of reuterin was also studied in the present invention, wherein glutaraldehyde was again used as a control. The cytotoxicity of the test reagents (glutaraldehyde vs. reuterin) was evaluated in vitro using a mouse-derived established cell line of 3T3 fibroblasts (BALB/3T3 C1A31-1-1). The assay (light microscopic observation and MTT assay) was used to measure the proportion of viable cells following a test-reagent-treated culture.

In the assay, 3T3 fibroblasts were seeded in 24-well plates at 5×10⁴ cells/well in 1 ml Dulbecco's modified eagle medium (DMEM, Gibco 430-2800EG, Grand Island, N.Y., USA) with 10% fetal calf serum (FCS, Hyclone Laboratories, Logan, Utah, USA). The cell culture was maintained in a humidified incubator at 37° C. with 10% CO₂ in air. Cells in log phase of growth were then exposed to a new DMEM medium drugged with varying concentrations of glutaraldehyde or reuterin. After 24 h of culture, the growth media in the wells were removed and the cells were photographed using light microscopy. Subsequently, the cells were washed with phosphate buffered saline (PBS) twice and surviving cell numbers were then determined indirectly by 3-(4,5-dimethylthiazol-yl)-2,5-diphenyltetrazolium bromide (MTT, Sigma Chemical Co., St. Louis, Mo., USA) dye reduction.

The MTT assay is based on the reduction of MTT, a yellow soluble dye by the mitochondrial succinate dehydrogenase to form an insoluble dark blue formazan product. Only viable cells with active mitochondria reduce significant amounts of MTT to formazan. In the test, 200 μl MTT solution (0.5 g/l in medium, filter-sterilized) was added to the culture wells. After incubation for 3 h at 37° C. in a 10% CO₂ atmosphere, the MTT reaction medium was removed and blue formazan was solubilized by 100 μl dimethylsulfoxide (DMSO). Optical density readings were then performed using a multiwell scanning spectrophotometer (MRX Microplate Reader, Dynatech Laboratories Inc., Chantilly, Va., USA) at a wavelength of 570 nm.

A photomicrograph of the 3T3 fibroblasts cultured in the medium without any test crosslinking reagent showed the cells cultured in the control medium were confluent, which may be used as our control in the evaluation of the cytotoxicity of glutaraldehyde and reuterin. Photomicrographs of the 3T3 fibroblasts cultured in the media drugged with varying concentrations of glutaraldehyde or reuterin revealed that: a) the cells cultured in the medium drugged with an extremely low concentration of glutaraldehyde (0.05 ppm) were confluent; b) as the concentration of glutaraldehyde increased to 5 ppm, all the cells cultured were found dead; and c) in contrast, as the concentration of reuterin increased to 15 ppm, the cells cultured were confluent.

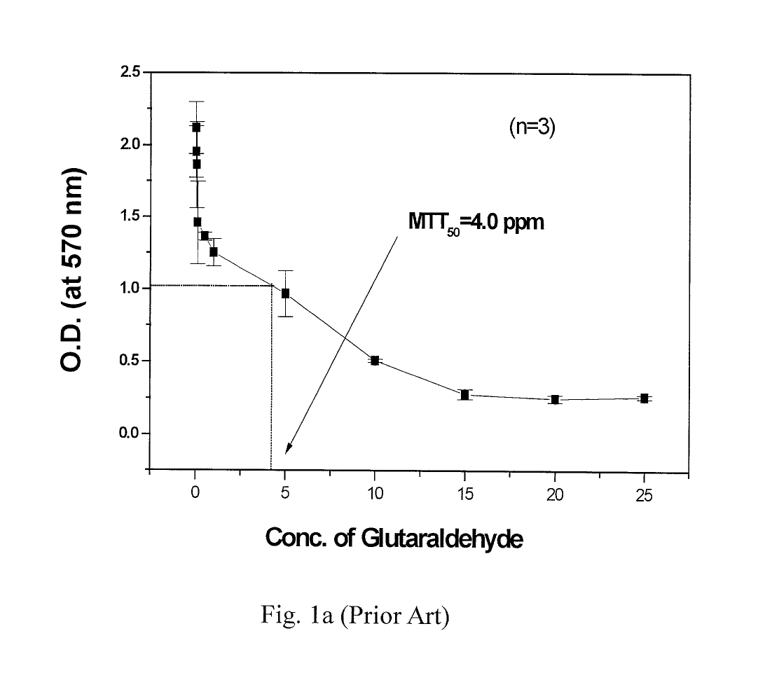

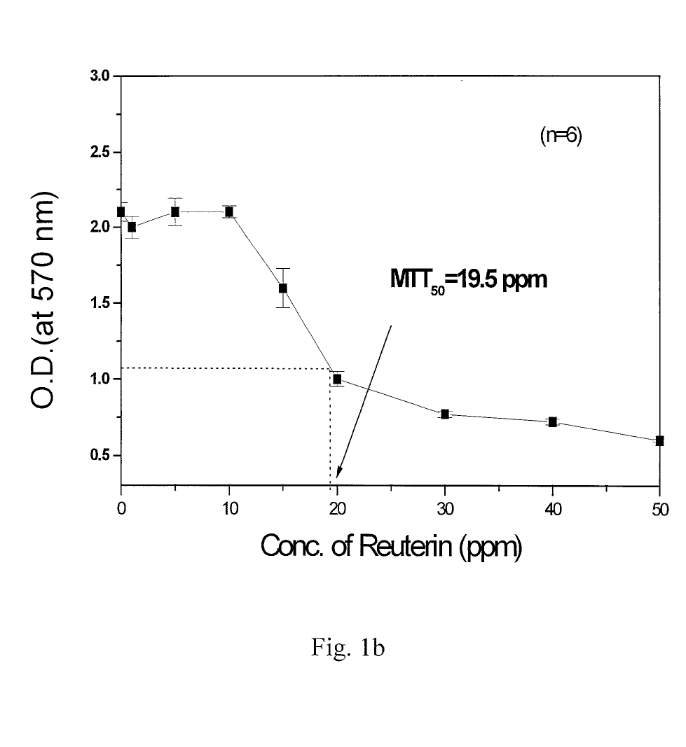

FIGS. 1a and 1b illustrate the optical density readings of the 3T3 fibroblasts cultured in the media drugged with varying concentrations of glutaraldehyde or reuterin obtained in the MTT assay. As shown in the figures, the optical density reading of the cells cultured in the medium drugged with glutaraldehyde declined more remarkably than that drugged with reuterin, as the concentration of the test reagent increased. The MTT₅₀ concentration of glutaraldehyde was approximately 4 ppm, which was much lower than that of reuterin (˜20 ppm).

### Example 1: Fixation of Biological Tissues

Materials and Methods

In this example, fresh porcine pericardia procured from a slaughter house were used as raw materials. The procured pericardia were transported in a cold physiological saline solution. Upon return, the pericardia first were gently rinsed with fresh saline to remove excess blood on the tissue. Adherent fat then was carefully trimmed from the pericardial surface. The maximum time period between retrieval and initiation of tissue fixation was less than 6 hours.

In the first part of this example, the rate of tissue fixation by reuterin was investigated. Glutaraldehyde was used as a control. The trimmed pericardia first were fixed in a 0.068M aqueous glutaraldehyde or reuterin solution buffered with phosphate-buffered saline (PBS, pH 7.4) at room temperature (25° C.). The amount of solution used in each fixation was approximately 100 mL for a 6-×6-cm porcine pericardium. Samples of each studied group then were taken out at distinct elapsed fixation duration periods (at 5 min, 1 h, 4 h, 12 h, 24 h, 48 h, and 72 h after the initiation of tissue fixation, respectively). The rate of tissue fixation by reuterin was determined by monitoring the changes in fixation index and denaturation temperature of the fixed tissues during the course of fixation.

In the second part of this example, the effects of fixation conditions (pH, temperature, and initial fixative concentration) on the degrees of tissue fixation by reuterin were investigated. The degree of tissue fixation by reuterin was determined by measuring the crosslinking characteristics (fixation index and denaturation temperature) of the fixed tissue. To elucidate the effects of pH on the degree of tissue fixation by reuterin, a 0.068M aqueous reuterin solution was buffered with: citric acid/sodium citrate (pH 4.0); PBS (pH 7.4); sodium borate (pH 8.5); or sodium carbonate/sodium bicarbonate (pH 10.5) at room temperature (25° C.). The effects of temperature on the degree of tissue fixation by reuterin were evaluated at: 4° C., 25° C., 37° C., or 45° C. A 0.068M aqueous reuterin solution buffered at pH 7.4 was used. To elucidate the effects of initial fixative concentration on the degree of tissue fixation by reuterin, a 0.034M, 0.068M, 0.1M, or 0.2M aqueous reuterin solution buffered at pH 7.4 at 25° C. was used. The duration for each fixation was 72 h.

The fixation index, determined by the ninhydrin assay, was defined as the percentage of free amino groups in tissue reacted with the test crosslinking agent subsequent to fixation. In the ninhydrin assay, the test tissue first was lyophilized for 24 h and then weighed. Subsequently, the lyophilized tissue was heated with a ninhydrin solution for 20 min. After heating with ninhydrin, the optical absorbance of the solution was recorded with a spectrophotometer (Model UV-150-02, Shimadzu Corp., Kyoto, Japan) using glycine at various known concentrations as standard. It is known that the amount of free amino groups in the test tissue, after heating with ninhydrin, is proportional to the optical absorbance of the solution. The denaturation temperature of each studied group was measured in a Perkin-Elmer differential scanning calorimeter (Model DSC 7, Norwalk, Conn.). This technique was widely used in studying the thermal transitions of collagenous tissues.

Results

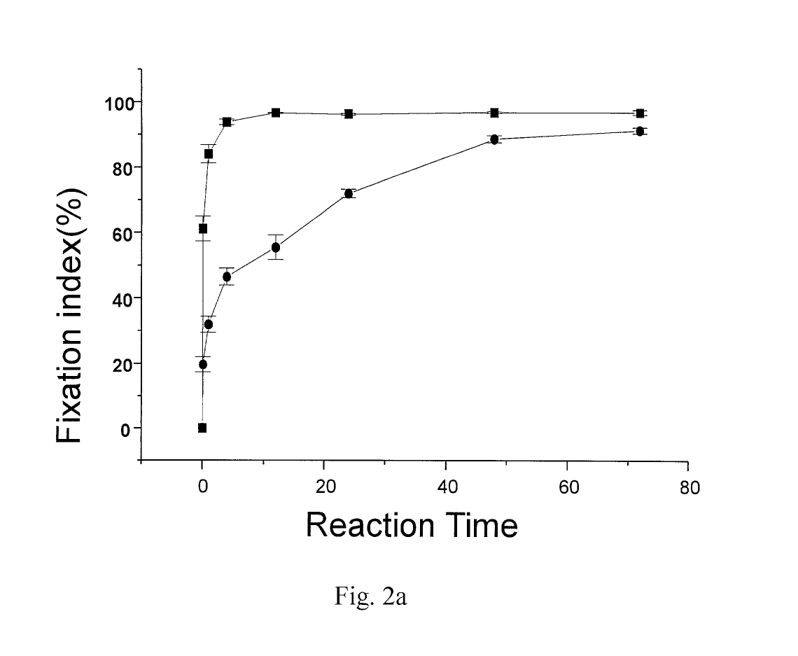

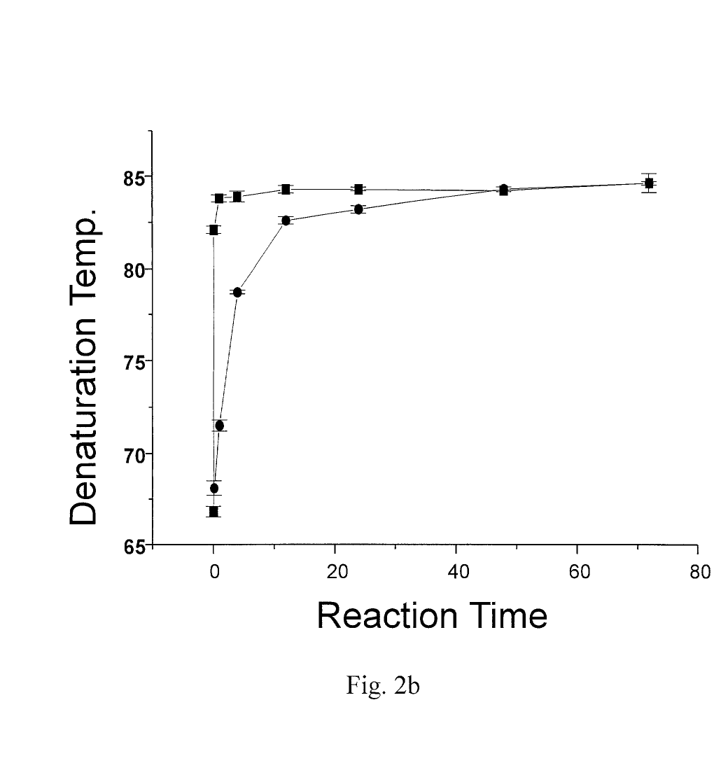

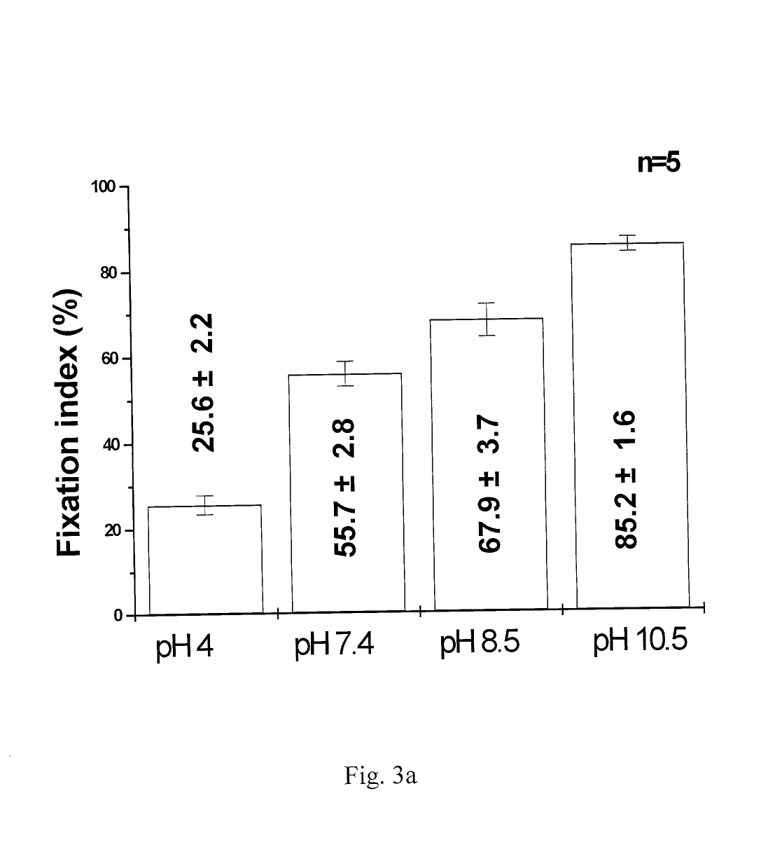

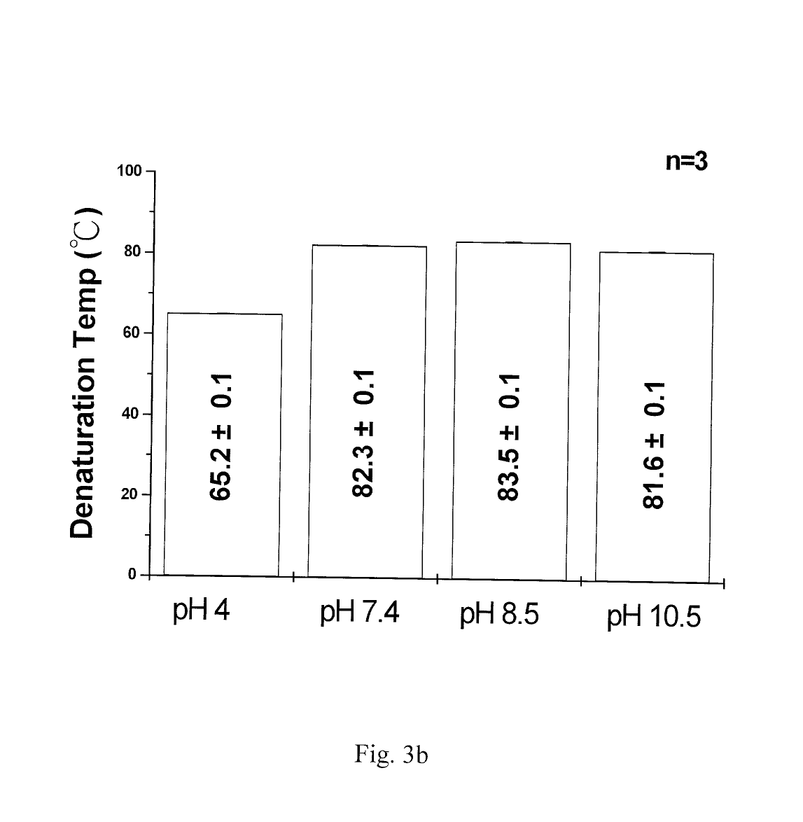

FIGS. 2a and 2b compare the fixation indices and denaturation temperatures of the tissues fixed with glutaraldehyde or reuterin obtained at various elapsed fixation duration periods. As shown in FIGS. 2a and 2b, both the fixation index and denaturation temperature of the glutaraldehyde-fixed tissue increased more rapidly than the reuterin-fixed tissue at the beginning of fixation. However, after 48 h of fixation, the fixation index and denaturation temperature of both studied groups were comparable. The pH of the buffer used in fixation played an important role in affecting the crosslinking characteristics of the reuterin-fixed tissue. FIGS. 3a and 3b present the fixation indices and denaturation temperatures of the tissues fixed by reuterin under various pHs. In general, the fixation indices of the reuterin-fixed tissues increased with increasing the fixation pH value. The denaturation temperatures of the tissues fixed by reuterin at pH 7.4 or pH 8.5 were relatively greater than that fixed at pH 10.5, while the tissue fixed at pH 4.0 had the lowest fixation indices and the lowest denaturation temperature.

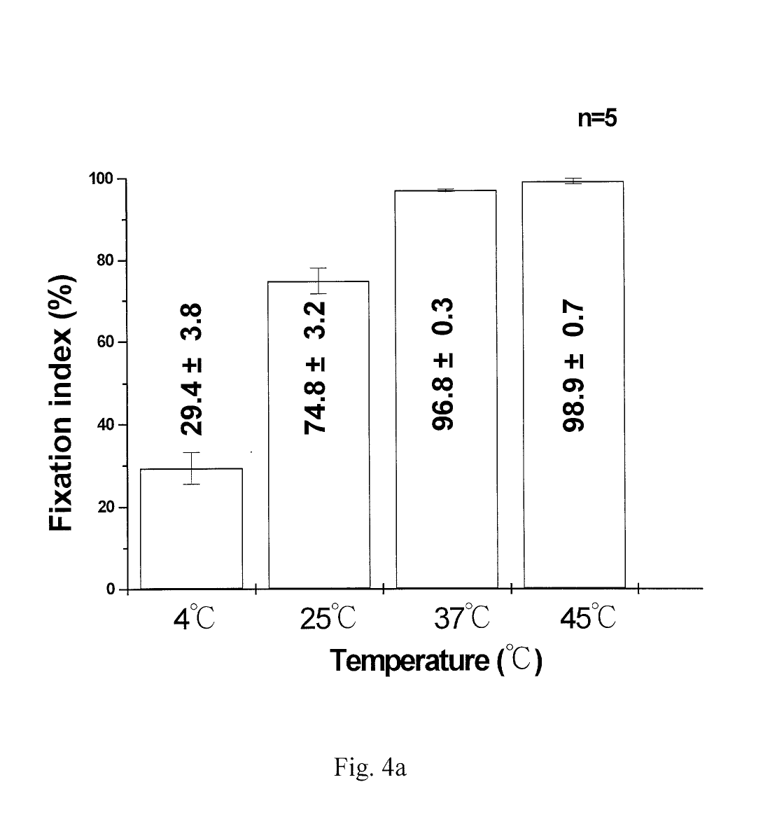

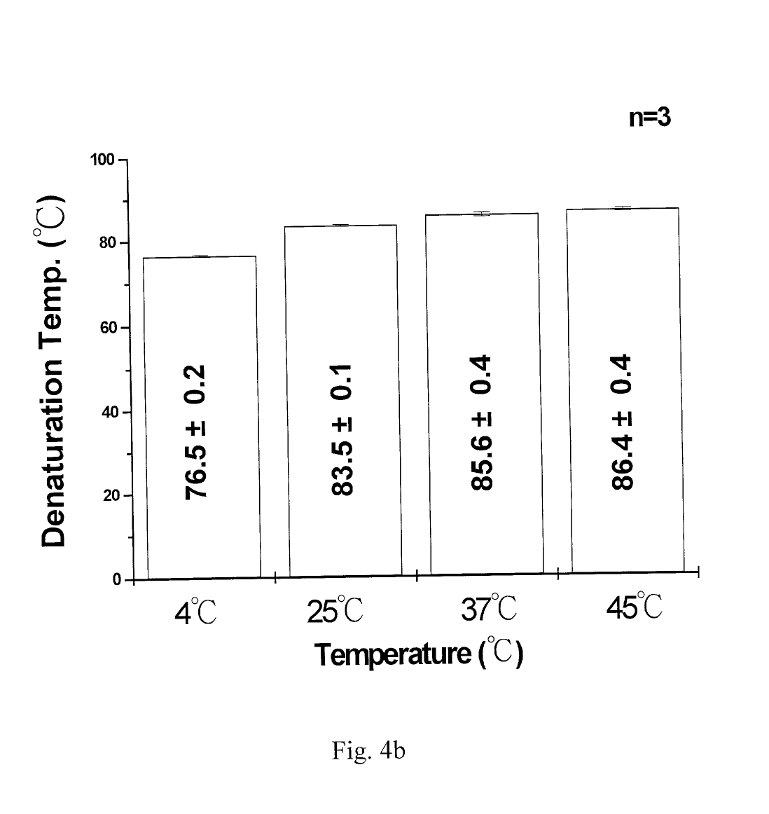

The fixation temperature significantly influenced the crosslinking characteristics of the reuterin-fixed tissue. The effects of temperature on the fixation index and denaturation temperature of the reuterin-fixed tissue are presented in FIGS. 4a and 4b. As indicated in FIGS. 4a and 4b, the tissues fixed at 37° C., or 45° C. had comparable fixation indices and denaturation temperatures. In contrast, the tissue fixed at 4° C. had the lowest fixation index and the lowest denaturation temperature among all groups studied at different temperatures.

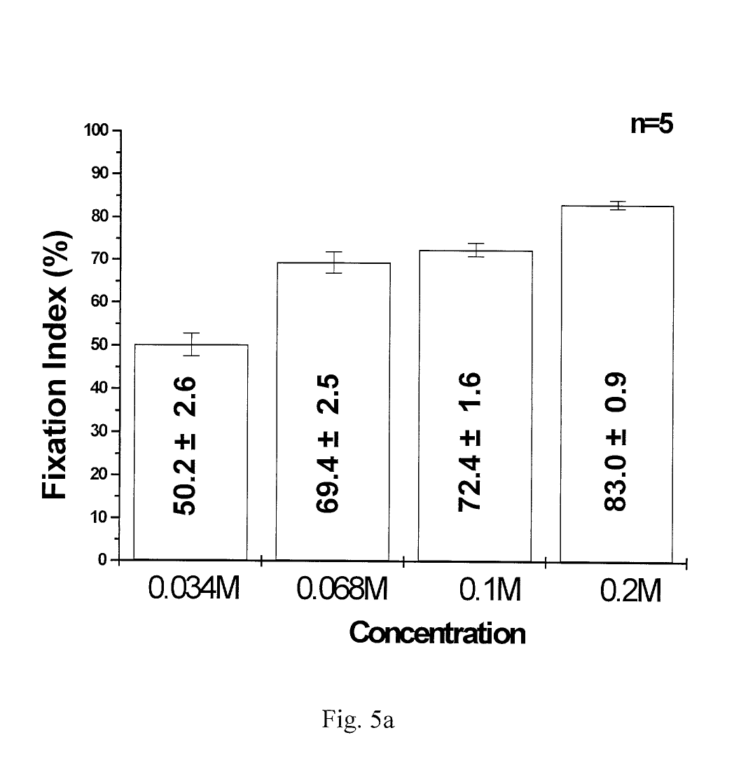

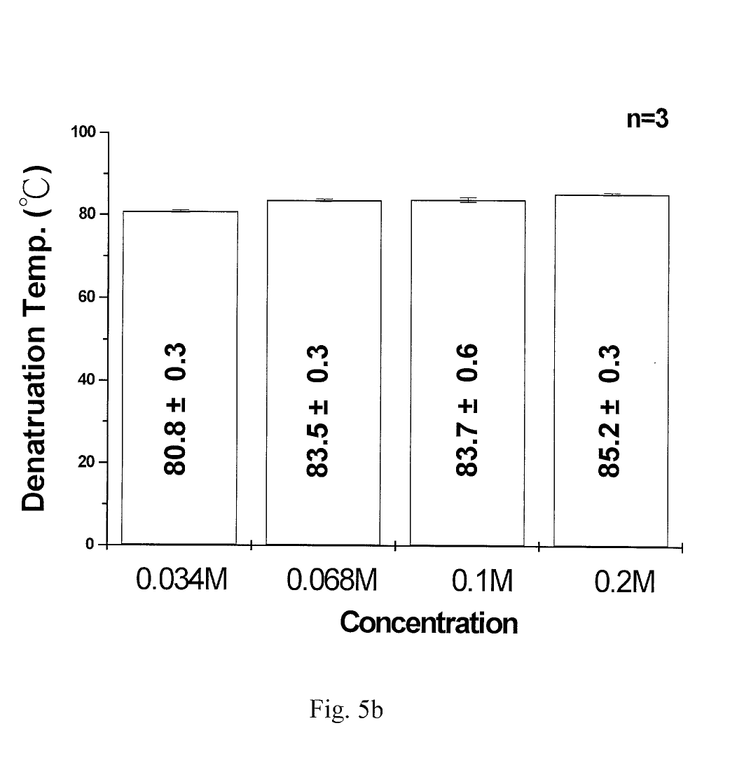

The effects of initial fixative concentration on the crosslinking characteristics of the reuterin-fixed tissue are given in FIGS. 5a and 5b. As given in FIGS. 5a and 5b, the fixation indices increased with increased initial fixative concentrations and denaturation temperatures of the tissues fixed by reuterin at different initial fixative concentrations were approximately equivalent.

### Example 2: Biocompatibility Study and Subcutaneous Study

To evaluate the biocompatibility of the biological tissues fixed with reuterin, a subcutaneous study was conducted using a growing rat model. Fresh and the glutaraldehyde-fixed counterparts were used as controls.

Materials and Methods

Fresh porcine pericardia was used as raw materials and treated as in Example 1.

The trimmed pericardia were fixed in a 0.068M glutaraldehyde or genipin solution at 37 ° C. for 3 days. The amount of solution used in each fixation was approximately 200 mL for a 6- ×6-cm porcine pericardium. The reuterin solution was buffered with sodium borate (pH 8.5), whereas the glutaraldehyde solutions were buffered with phosphate buffered saline (0.01M pH 7.4). After fixation the test samples were divided into two groups. For the first group, the fixed pericardia were rinsed in sterilized phosphate buffered saline with a solution change for several times for approximately 5 hrs. For the second group, the fixed pericardia were sterilized with a series of ethanol solutions in an order of increasing concentration (20˜75%) for approximately 5 hrs.

Subsequently, the test samples were implanted subcutaneously in a growing rat model (6-week-old male Wistar) under aseptic conditions. The implanted samples were retrieved at 3 days and 1, 4, and 12 weeks following the procedures. The denaturation temperatures of the retrieved samples were determined by a differential scanning calorimeter (Perkin Elmer Model DSC 7, Norwalk, Conn., USA). The content of calcium deposited on each retrieved sample was assessed with atomic absorption spectroscopy.

Results

In the gross examination, it was found that fresh samples were thinner than the other fixed samples at 1-week post implantation. At 4-week postoperatively, fresh samples were completely degraded, while the other fixed samples remained intact.

It was found that the denaturation temperatures of the same studied group retrieved at different post implantation times were substantially the same. Of the fixed samples, the denaturation temperatures of the reuterin-fixed samples were comparable to their glutaraldehyde-fixed counterparts. The denaturation temperatures of the fixed samples were about 85° C., which was significantly greater than that (62° C.) of the fresh one.

The photomicrographs of the fresh, glutaraldehyde- and reuterin-fixed tissues stained with H&E retrieved at 3-day postoperatively showed that the fresh tissue had the most notable inflammatory reaction among all the studied groups. The degrees in inflammatory reaction observed for the glutaraldehyde- and reuterin-fixed tissues retrieved at this time were not significantly different. At 4-week postoperatively, the degree of inflammatory reaction for each studied group was more remarkable than its corresponding counterpart retrieved at 3-day postoperatively. As observed at 3-day postoperatively, the degrees in inflammatory reaction for the glutaraldehyde- and reuterin-fixed tissues were not significantly different.

The photomicrographs of the glutaraldehyde-, and reuterin-fixed tissues retrieved at 12-week postoperatively were also taken. It should be noted that no photomicrograph of the fresh tissue retrieved at this time could be made, due to its complete degradation. As observed in the photomicrographs, the degrees in inflammatory reaction for all the fixed samples were less notable than those retrieved at 1- and 4-week postoperatively. Of note is that the inflammatory cells surrounding the reuterin-fixed tissue were less than the glutaraldehyde-fixed tissue.

The results of the calcium contents for the fresh, glutaraldehyde-, and reuterin-fixed tissues before implantation and those retrieved at 3-day, 1-, and 4-week postoperatively are presented in Table I. It should be noted that no data could be obtained for the fresh tissues retrieved at 4-week postoperatively, due to their complete disintegration. As presented in the table, the difference in calcium content between the samples before implantation and those retrieved at distinct implantation duration were not significant for all the studied groups.

TABLE I
Calcium Contents (μg calcium/mg dry tissue weight)*
of Each Studied Group Before Implantation
and Retrieved at Distinct Implantation Duration

| Implantation Duration | Fresh | Glutaraldehyde | Reuterin |
|---|---|---|---|
| 0-week (n = 4) | 1.2 ± 0.1 | 1.4 ± 0.1 | 1.5 ± 0.3 |
| 3-day (n = 4) | 1.3 ± 0.1 | 1.5 ± 0.3 | 1.5 ± 0.2 |
| 1-week (n = 4) | 1.9 ± 0.2 | 2.1 ± 0.9 | 1.6 ± 0.3 |
| 4-week (n = 4) | N/A# | 1.8 ± 0.6 | 1.7 ± 0.5 |

*The numbers are presented in mean ± standard deviation.
#N/A: Data were not available, due to the complete degradation of the fresh tissues observed at 4-week postoperatively.

Additionally, the tensile strength of each retrieved sample was measured by an Instron Universal Testing, Machine (Model 4302) at a constant speed of 50 mm/min. The results showed the tensile strengths of the reuterin-fixed and glutaraldehyde-fixed samples were comparable before implantation and retrieved at distinct duration periods postoperatively.

Although the present invention has been described with reference to specific details of certain embodiments thereof, it is not intended that such details should be regarded as limitations upon the scope of the invention except as and to the extent that they are included in the accompanying claims. Many modifications and variations are possible in light of the above disclosure.

We claim:

1. A biocompatible implant, substitute or wound dressing comprising a crosslinked biomolecule formed by crosslinking an amine-containing biomolecule with 3-hydroxypropinoaldehyde.

2. The biocompatible implant, substitute or wound dressing according to claim 1, wherein said biomolecule is a connective-tissue protein.

3. The biocompatible implant, substitute or wound dressing according to claim 1, wherein said biomolecule is chitosan.

4. The biocompatible implant, substitute or wound dressing according to claim 2, wherein said connective-tissue protein is collagen or gelatin derived from a collagenous source.

5. The biocompatible implant, substitute or wound dressing according to claim 1, wherein said biomolecule is hemoglobin.

6. In a method of manufacturing a biocompatible implant, substitute or wound dressing, the improvement comprising crosslinking an amine-containing biomolecule with 3 -hydroxypropinoaldehyde.

7. The method according to claim 6, wherein said biomolecule is a connective-tissue protein.

8. The method according to claim 6, wherein said biomolecule is chitosan.

9. The method according to claim 7, wherein said connective-tissue protein is collagen or gelatin derived from a collagenous source.

10. The method according to claim 6, wherein said biomolecule is hemoglobin.

11. The method according to claim 6, wherein said crosslinking comprising contacting said biomolecule and 3-hydroxypropinoaldehyde in an aqueous medium at a temperature ranging from 4° C. to 50° C. for a period ranging from 5 hours to 60 hours.

12. The method according to claim 1 1, wherein said aqueous medium has a concentration of 3-hydroxypropinoaldehyde ranging from 0.01 M to 1.0 M.

13. The method according to claim 11, wherein said aqueous medium has a pH value ranging from 3 to 12.

14. The method according to claim 11, wherein said temperature ranges from 25° C. to 45° C.

15. The method according to claim 11, wherein said period is about 48 hours.

16. The method according to claim 12, wherein said aqueous medium has a concentration of 3-hydroxypropinoaldehyde ranging from 0.03 M to 0.2 M.

17. The method according to claim 13, wherein said aqueous medium has a pH value ranging from 4 to 10.5.

18. A method for treating a patient requiring tissue prosthesis comprising crosslinking an amine-containing biomolecule with 3-hydroxypropinoaldehyde, and implanting a biocompatible implant or wound dressing comprising the resulting crosslinked biomolecule into said patient.

19. A method for conducting blood transfusion in a patient comprising crosslinking hemoglobin with 3-hydroxypropinoaldehyde and transfusing a blood substitute comprising the resulting crosslinked hemoglobin to said patient.
